(19) United States
(12) Patent Application Publication
(10) Pub. No.: US 2007/0122808 A1
(43) Pub. Date: May 31, 2007

(54) Measurement of a polynuleotide amplification reaction

(76) Inventor: Daniel Henry Densham, Exeter (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/564,792

(22) PCT Filed: Jul. 15, 2004
(86) PCT No.: PCT/GB04/03086

§ 371 Date: Jun. 5, 2006

(30) Foreign Application Priority Data
Jul. 15, 2003 (GB) 0316555.2
Dec. 8, 2003 (GB) 0328425.4

(51) Int. Cl. C12Q 1/68; C12P 19/34

(52) U.S. Cl. 435/6; 435/91.2

(57) ABSTRACT

A quantitative measurement of the progress of a polynucleotide amplification reaction can be made by: (i) carrying out a reaction for the amplification of a target polynucleotide; (ii) either during or after the amplification reaction contacting the amplified product with a molecule that binds to or interacts with a polynucleotide, the molecule being located in a spatially defined position or being determined via a non-linear or non-fluorescent technique; and (iii) detecting the interaction between the amplified product and the molecule by measuring changes in radiation.

### FIELD OF THE INVENTION

The present invention relates to monitoring polynucleotide amplification reactions.

### BACKGROUND TO THE INVENTION

The ability to generate multiple copies of a particular polynucleotide in an amplification reaction is important in many commonplace biotechnology processes. The polymerase chain reaction (PCR), as disclosed in U.S. Pat. No. 4,683,202, permits exponential amplification of a polynucleotide to achieve large quantities of the polynucleotide. In its simplest form, PCR is an in vitro method for the enzymatic synthesis of specific DNA sequences, using two oligonucleotide primers that hybridise to opposite strands and flank a region of interest in the target DNA. A repetitive series of reaction steps involving template denaturation, primer annealing, and the extension of the annealed primers by DNA polymerase results in the exponential accumulation of the target polynucleotide.

At the start of a PCR reaction, reagents are in excess and the template and product are at a sufficiently low concentration so that product denaturation does not compete with primer binding, and the amplification reaction proceeds at a constant exponential rate.

There are many diagnostic assays that utilise PCR and rely on the quantification of the amplified products. For accuracy and precision, it is necessary to collect quantitative data at a point at which the sample is in the exponential phase of amplification (this is important as it is the exponential phase that provides reproducible results). The need to monitor the amplification reaction can slow down the time taken to complete the diagnostic assay due to the need to take samples of the amplification products and determine the quantity of amplified products present at different time stages.

Real-time PCR automates this process by quantitating reaction products for each sample in every amplification cycle. This reaction relies upon the detection and quantification of a fluorescent reporter molecule, the signal of which increases in direct proportion to the amount of amplified product in a reaction.

There are now many commercially available real-time PCR kits which rely on particular fluorescent molecules.

Although real-time PCR has increased sensitivity and dynamic range over traditional (end point) PCR, there are a number of inherent difficulties relating to the use of this system. The disadvantages are due primarily to the use of the fluorescent labels required in real-time PCR techniques and/or the way in which such labels are used. The fluorescent labels must be highly chemically stable, both in terms of the amount of excitation light they can absorb and their ability to withstand the temperature required in the PCR process. The number of dyes available for such a process is therefore restricted and affects the amount of multiplexing possible. Further, for successful multiplexing in prior art fluorescent based systems, each dye in the set must be spectrally resolvable from the other dyes. It is difficult to find a collection of dyes whose emission spectra are spectrally resolved, since the typical emission band half-width for organic fluorescent dyes is about 40-80 nanometers and the width of the available spectrum is limited by the excitation source. The fluorescent signal of each dye must also be sufficiently strong so that each component can be detected with sufficient sensitivity. The use of fluorescent labels also limits the polynucleotide sequence that may be used as a probe for attachment to the amplified product, as guanine residues are known to act as quenchers in a fluorescent resonant energy transfer process and therefore care must be taken when selecting the sequences immediately adjacent to the position at which the fluorescent molecule is attached to the amplified product.

The limited number of fluorescent molecules available and the common use of a monochromatic energising light source also limits the extent to which multiplexed real-time PCR can be carried out, i.e. there is a limit to the number of different polynucleotides that may be amplified and detected in a single reaction.

There is therefore a need for an improved method for monitoring the amplification process. In particular, there is a need for a method which can be used to carry out highly multiplexed reactions in an automated process.

### SUMMARY OF THE INVENTION

The present invention is based on the realisation that the progress of an amplification reaction may be monitored by detecting the interaction between an amplified product and a molecule that interacts with or binds to the amplified product and whose identity is spatially defined and/or determined via a non-linear/non-fluorescent technique.

According to a first aspect of the present invention, a method for monitoring a polynucleotide amplification reaction comprises the steps of:
(i) carrying out a reaction for the amplification of a target polynucleotide;
(ii) either during or after the amplification reaction contacting the amplified product with a molecule that binds to or interacts with a polynucleotide, the molecule being located in a spatially defined position or being identified via a non-linear or non-fluorescent technique; and
(iii) detecting the interaction between the amplified product and the molecule by measuring changes in applied radiation.

The method of the invention can be carried out without the requirement for fluorophores and therefore overcomes the disadvantages associated with the use of fluorophores. Alternatively, if fluorophores are used, an increase in the level of multiplexing may be achieved by utilising a polynucleotide-binding molecule located in a spatially defined position. In addition, the method of the invention can be carried out on a real-time basis, without the need to obtain samples during the amplification process. Real time multiplexed monitoring of an amplification reaction can therefore be achieved.

### DESCRIPTION OF THE DRAWINGS

The invention is described with reference to the accompanying figures, wherein:

FIG. 1 is a schematic illustration of a “bulk” refractive index compensated SPR biosensor; and

FIG. 2 shows the compensated correlation shift of hybridisation and subsequent thermal “melting” of a complementary polynucleotide.

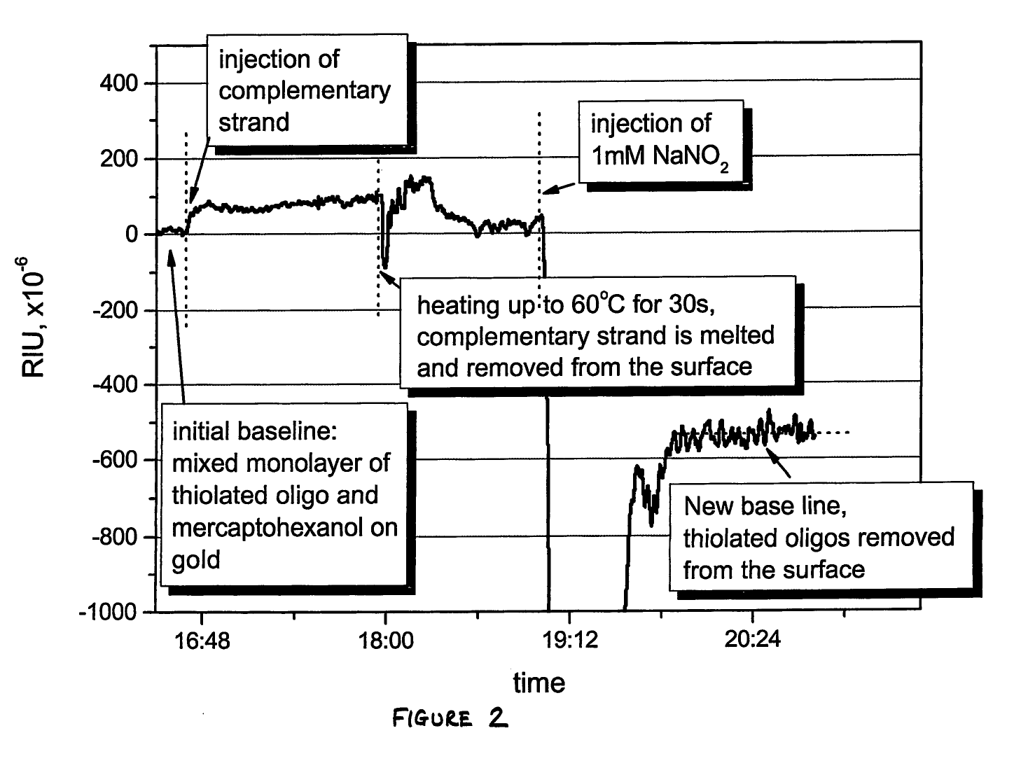

### DESCRIPTION OF THE INVENTION

The present invention provides a way of monitoring the progress of a polynucleotide amplification reaction involving the analysis of the interaction between an amplified polynucleotide and a molecule that interacts with or binds to a polynucleotide.

The term “polynucleotide” as used herein is to be interpreted broadly, and includes DNA and RNA, including modified DNA and RNA, as well as other hybridising nucleic acid-like molecules, e.g. peptide nucleic acid (PNA). The term encompasses oligonucleotides which comprise short sequences of nucleic acid monomers.

The present invention relies on the use of a molecule that binds to or otherwise interacts with a polynucleotide. The molecule may be any molecule that binds to a polynucleotide in a specific or non-specific manner. The molecule may interact with a polynucleotide which is in a double-stranded or single-stranded form. Molecules which interact with polynucleotides will be apparent to the skilled person. In one embodiment, the molecule is a protein and may be a DNA or RNA-binding protein. Suitable proteins may be recombinant proteins which have been modified to contain a site-specific polynucleotide binding domain. Such domains are well known in the art, and are disclosed in Duncan et al., Genes Dev., 1994; 8(4): 465-80. Examples of proteins which interact with polynucleotides and which are therefore within the scope of the present invention include: helicases, transcriptases, primases and histones.

In a particularly preferred embodiment, the molecule is a polymerase enzyme which may be utilised in the amplification reaction. Accordingly, the detection of the interaction may be carried out at the same time as the amplification reaction proceeds.

In this embodiment, the primers for the amplification reaction maybe labelled. Preferred labels include, but are not limited to, Raman scattering labels (.e.g. those outlined in U.S. Pat. No. 6,514,767). Alternative labels will be apparent to those skilled in the art. Such labels include organic and non-organic fluorescent dyes, e.g. metal particles. In this embodiment, therefore, during the polymerisation reaction, primers are brought into close proximity to the surface to which the polymerase molecules are attached due to the formation of a molecular complex. This proximity to a surface assists in the detection of primers involved in the amplification reaction and hence readout. Techniques involving the use of Evanescent fields are a preferred embodiment of the present invention. In the case of Raman scattering, the proximity to a surface will enhance detection.

The use of evanescent field excitation techniques provides the benefit of a reduction in background noise due to the exponential decay of the field away from the surface.

When Raman scattering labels are employed, the surface may be of a patterned ‘free electron’ metal surface, such that local raman scattering intensity is increased further. Alternatively, the metal layer may be any that supports Surface Plasmon Resonance (SPR).

In a foreseen embodiment in which immobilised polymerases are used, ‘conventional’ FRET based real-time PCR labelling techniques are employed. In this way detection is simplified and background is reduced. Such labelling schemes include the nuclease and Taqman assays which are available commercially. Other non-FRET based fluorescent dye systems are also within the scope of the present invention.

In a further embodiment, the molecule is a single-stranded polynucleotide having a sequence (or part of the sequence) that is complementary to at least part of the amplified polynucleotide. In this embodiment, a plurality of such polynucleotides can be immobilised on a support material and hybridisation of the amplified polynucleotides onto the immobilised polynucleotides can be monitored by monitoring the change in applied radiation which occurs on hybridisation. This sequence may or may not be one which takes part in the amplification reaction.

The arrayed polynucleotides may be applied to substrate via a number of techniques known to one skilled in the art. For example, approaches for making polynucleotide arrays for gene expression are generally applicable.

There are two general approaches to making polynucleotide arrays. These are photolithographic methods and “contact” arraying methods. In photolithographic methods, synthetic linkers modified with photochemically removable protecting groups are attached to a silicon surface. Light is directed through a photolithographic mask, removing the protecting groups from specific parts of the surface. The surface is then incubated with one of the four hydroxyl-protected deoxynucleotides, which couple with the polynucleotide strands that have been deprotected. A new mask is then used, so that different parts of the surface are illuminated. The cycle is then repeated until the strands are the desired length and sequence. With “contact” arraying methods, polynucleotides are physically printed onto the desired surface, commonly a glass microscope slide that has been coated with poly-l-lysine to make the polynucleotide stick to the surface.

When probe molecules are arrayed on a support surface, their spatial location may be used to define their identity as with conventional expression arrays. The molecules may therefore be single molecules that are immobilised on the support so that they can be identified individually, or different types of molecules may be positioned in separate regions of the support.

In a preferred embodiment of the present invention, immobilised probe molecules are arrayed on a support material and the amplification reaction is carried out in the same enclosed vessel as the array. Hybridisation between amplified products and complimentary sequences is then monitored either throughout or at set points within the amplification reaction (i.e. between the same temperatures during subsequent amplification cycles) in order to obtain quantitative information on the concentration of the polynucleotide of interest. In this embodiment, a number of techniques for detection of hybridisation may be applied as will be apparent to those skilled in the art. For example, an intercalating dye may be used. Such dyes are typically speaking, flat aromatic molecules that bind non-covalently to double-stranded DNA or RNA by positioning themselves between adjacent base pairs of the duplex. Intercalating fluorescent dyes are generally non-fluorescent in solution, becoming fluorescent when binding with double-stranded polynucleotides (e.g. U.S. Pat. No. 6,472,153). Thus, in fluorescent mode read-out, the present invention may be achieved with a number of commercially available dyes and dye systems for the detection of polynucleotide hybridisation. Intercalating dyes are a particularly preferred embodiment due to the low cost and availability. Spectral overlap is not an issue in this case due to the spatial separation of the probe locations.

In a Raman embodiment of the spatially defined approach to multiplexing, Raman supporting particles are bound to intercalating molecules. In a preferred embodiment, the intercalating molecules are covalently bound to a Raman-supporting particle or particles via a linker molecule such that the intercalating molecule may still form a complex with the double stranded polynucleotide. Such linker molecules will be apparent to those skilled in the art.

In a further embodiment, detection of hybridisation between the immobilised probe molecule and the amplified product is carried out via the use the monitoring of changes of electrical conductance and/or capacitance. Such arrays based upon electrical properties at array locations exist within the state of the art and are within the scope of the present invention.

In one embodiment, the molecule is a short polynucleotide that functions as the primer molecule for the initiation of the amplification reaction. According to this embodiment, the original target polynucleotide or amplified product is brought into contact with the primer molecule under conditions suitable for hybridisation to occur. The interaction between the primer molecule and the target can be monitored prior to or during the amplification reaction, so that a quantitative measurement can be taken. Products amplified from the reaction will also hybridise to other “free” primer molecules, to thereby initiate a further amplification reaction. In this embodiment, the solution phase primer molecules should be present in large excess, as primer extension will prevent the re-use of the molecules as primers.

Immobilised oligonucleotides may also be used as probes, to bind to the amplified products, with detection of the interaction being carried out as the amplification reaction proceeds.

The molecule required for interacting with the target polynucleotide (or amplified product) may comprise a label, which enhances the detection of applied radiation. For example, if the detection method is based on applying surface electromagnetic waves (e.g. SPR) a plasmon-supporting particle may be attached to the molecule to enhance the generated signal. Suitable particles include, gold spheres (nanoparticles).

Attachment of the label may be via direct covalent attachment to the molecule or indirect attachment. For example, the amplification reaction may be carried out such that the amplified product incorporates a suitable label. This may be achieved by utilising a labelled primer for initiating the amplification reaction. Techniques for the attachment of labels to polynucleotides are apparent to those skilled in the art.

It will be usual to utilise a plurality of types of molecules, so that multiple interactions can be detected.

The molecule that interacts with the target (or the amplification product), is preferably immobilised on a support material. This allows the detection to be carried out on fixed positions, and is important for those detection techniques which utilise a specific substrate to generate a detectable signal.

Techniques for immobilising molecules to support materials are known to those skilled in the art. Suitable support materials will also be known, and the choice of a suitable material may depend on the detection technique, as many techniques that monitor changes in radiation require specific substrates. For example, in the case of Raman enhancement, surfaces of suitably roughened gold, silver, copper or other free-electron metals, may be employed. Other such substrates include those that support surface enhancement of the Raman activity. Such activity is observed with metal colloidal particles, metal films on dielectric substrates, and with metal particle arrays.

In the case of fluorescent measurements, glass or silicon substrates are preferred, although a large number of substrates are compatible with the use of fluorescence. For use with surface plasmon resonance, gold, silver or other free electron containing substrates that are able to support a plasmon are considered within the scope of the present invention.

The detection of the interaction between an amplified product and the molecule is carried out by measuring changes in applied radiation. Measuring the changes in radiation that occur on interaction between an amplified product and the molecule may be carried out using conventional apparatus.

Non-linear imaging systems are known in the art. In general, the non-linear polarisation for a material can be expressed as:
P=X⁽¹⁾E¹+X⁽²⁾E²+X⁽³⁾E³+. . .
where P is the induced polarisation, X(n) is the nth-order non-linear susceptibility, and E is the electric field vector. The first term describes normal absorption and reflection of light; the second describes second harmonic generation (SHG), sum and difference frequency generation; and the third describes light scattering, stimulated Raman processes, third harmonic generation (TGH), and both two- and three-photon absorption.

A preferred imaging system of the present invention relies on the detection of the signal arising from second or third harmonic generation.

Single-molecule resolution using second or third harmonic generation (hereinafter referred to as SHG) is known in the art (Peleg et al., Proc. Natl. Acad. Sci. USA, 1999; 95:6700-6704 and Peleg et al., Bioimaging, 1996; 4:215-224). Suitable systems are described in WO 02/095070.

In one embodiment, detection is carried out in solution phase (i.e. the molecules are not immobilised), using raman scattering and/or LSPR techniques.

When light is directed onto a molecule, the vast majority of the incident photons are elastically scattered without a change in frequency. This is termed Rayleigh scattering. However, the energy of some of the incident photons (approximately 1 in every 10⁷ photons) is coupled into distinct vibrational modes of the molecule's bonds. Such coupling causes some of the incident light to be inelastically scattered by the molecule with a range of frequencies that differ from the range of the incident light. This is termed the Raman effect. By plotting the frequency of such inelastically scattered light against intensity, the unique Raman spectrum of the molecule under investigation is obtained. Analysis of the Raman spectrum of an unknown sample can yield information about the samples molecular composition.

The incident illumination for Raman spectroscopy, usually provided by a laser, can be concentrated to a small spot if the spectroscope is built with the configuration of a microscope. Since the Raman spectrum scales linearly with laser power, light intensity at the sample can be very high in order to optimise sensitivity of the instrument. Moreover, because the Raman response of a molecule occurs essentially instantaneously (without long-lived highly energetic intermediate states), photobleaching of the Raman-active molecule—even by this high intensity light—is impossible.

The Raman effect can be significantly enhanced by bringing the Raman active molecule(s) close (<50 Å) to a structured metal surface, this field decays exponentially away from the surface. Bringing molecules in close proximity to metal surfaces is typically achieved through adsorption of the Raman-active molecule onto suitably roughened gold, silver copper or other free-electron metals. Surface enhancement of the Raman activity is observed with metal colloidal particles, metal films on dielectric substrates, and with metal particle arrays. The mechanism by which this surface-enhanced Raman scattering takes place is not well understood, but is thought to result from a combination of (i) surface plasmon resonances in the metal that enhance the local intensity of the light, and ; (ii) formation and subsequent transitions of charge-transfer complexes between the metal surface and the Raman-active molecule.

Within a preferred embodiment of the present invention, Surface Enhanced Raman Scattering (SERS) is employed via the use of single gold or silver nanoparticles. In a preferred embodiment, single stranded polynucleotide probes are bound to single SERS nanoparticles. A Raman enhancing metal nanoparticle that has associated or bound to it a Raman-active molecule(s) can have utility as an optical tag. This general concept is outlined in U.S. Pat. No. 6,514,767, the content of which is hereby incorporated by reference. In one embodiment, if the target/probe of interest is immobilised on a solid support, then the interaction between a single target/probe molecule and a single (i.e. amplified) polynucleotide can be detected by searching the Raman active molecule's unique raman spectrum. Because a single Raman spectrum (from 100 to 3500 cm −1) can detect many different Raman-active molecules, SERS-active nanoparticles bound to or associated with polynucleotide binding molecules may be used in the context of the present invention within multiplexed assay formats.

In a preferred embodiment, changes in radiation are monitored by utilising surface electromagnetic wave technology.

Biosensors incorporating surface electromagnetic wave technology (and, in particular, surface plasmon resonance—SPR—sensors) are based on the sensitivity of surface electromagnetic waves (SEW) to the refractive index of the thin layer adjacent to the surface where the SEW propagates. In the biosensor, the amplified products are allowed to flow across the surface containing the immobilised molecule(s). As binding occurs, the accumulation or redistribution of mass on the surface changes the local refractive index that can be monitored in real time by the sensor.

Several methods utilising SPR technology have been proposed and realised in biosensors. The most popular methods are based on the Kretschmann-Raether configuration where the intensity of the light reflected from the sensor is monitored. This technique, considered to be one of the most sensitive, is described in J. Homola et al, Sensors and Actuators B 54, p. 3-15 (1999) and has a detection limit of 5×10⁻⁷ refractive index units. Measuring SPR phase changes can further increase the sensitivity of the sensor by one or two orders of magnitude. This is described in Nelson et al, Sensors and Actuators B 35-36, p. 187 (1996) and in Kabashkin et al, Optics Communications 150, p. 5 (1998). Prior art interferometric devices such as a Mach Zehnder device have been configured to measure variations in the refractive index at the sensor surface via phase shifts. This is disclosed in International Patent Publication WO-A-0120295. The configuration requires four independent components and is sensitive to sub-wavelength relative replacements of these components and hence very small mechanical and environment perturbations. A mechanically more robust monolithic interferometric design is outlined in WO-A-03014715.

In a preferred embodiment, a surface electromagnetic wave (SEW) sensor system is used which can compensate for changes in the bulk refractive index of a buffer or which allows the contribution of the bulk refractive index to an interference pattern to be separated from the contribution of an analyte absorbed on the sensor surface. The biosensor therefore comprises:
a coherent radiation source for producing an incident wave;
a carrier surface for supporting the immobilised molecule, the carrier surface mounted on a substrate and capable of supporting surface electromagnetic waves (SEW);
means for splitting the incident wave into an SEW and a first scattered wave, wherein the SEW propagates along the carrier surface and interacts with the immobilised molecule;
means for generating a second scattered wave from the SEW; and,
a detector for monitoring the interference between the first scattered wave and the second scattered wave.

In this embodiment, a coherent optical beam generated by a monochromatic laser is focussed using a lens, onto the edge of a metallic film able to support surface electromagnetic waves (SEWs). The optical beam passes through a glass prism on which the metallic film is mounted. A near-infrared laser is used as the illumination source. Using a near-infrared source has the advantage of long propagation length for surface plasmons in gold and silver while conventional optics can be still used for imaging and illumination. However, other monochromatic sources are suitable and may be used.

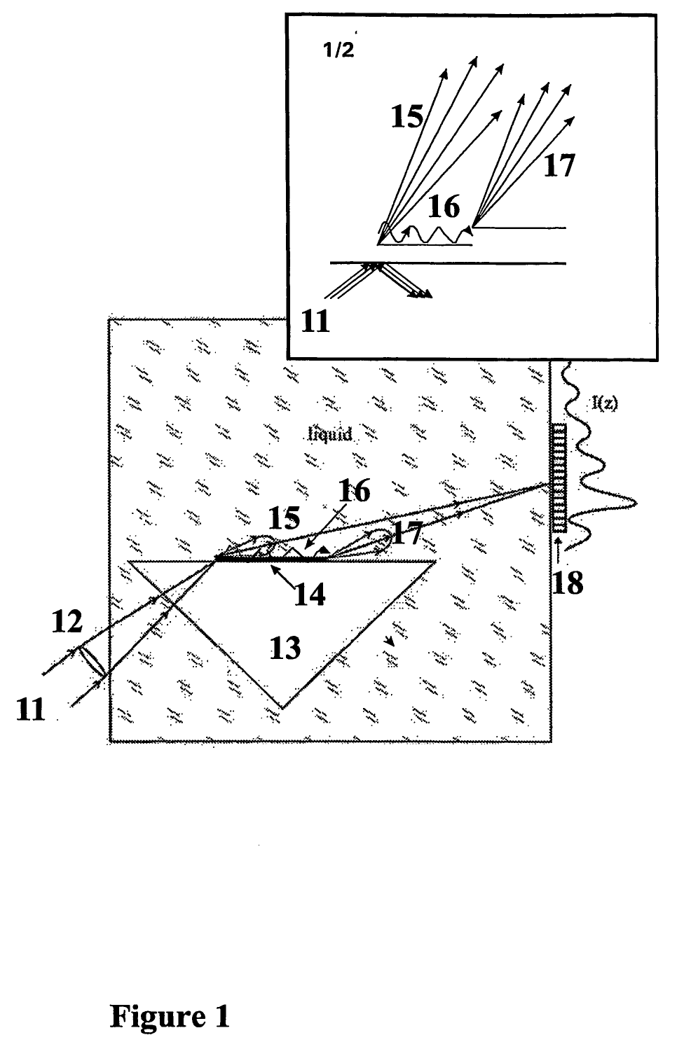

This is shown schematically in FIG. 1. The p-polarised near infrared laser beam 11 passes through the focusing lens 12 and then through the glass prism 13 where the substrate 14 with a microfabricated metal film is attached with an index matching liquid or gel. The glass prism may be a triangular prism as shown or a hemi-cylindrical prism. The angle of illumination is chosen slightly higher than the angle of total internal reflection on the interface substrate-solution. Being illuminated by the laser, the structure 14 generates scattered wave 15 and SEW 16 propagating along the metal-solution interface which is consecutively scattered into the volume wave 17. These waves propagate through the liquid cell and than produce an interference fringe pattern on the measurement device 18. Due to the fact that both scattered waves propagate through the solution, the contribution of bulk refractive index can be compensated by proper choice of experimental geometry.

The metal structure can be formed from gold or silver, or any other metal capable of supporting surface plasmons or a combination of them, or alternatively a dieletric multilayer supporting a SEW. It is preferred to use either a gold or silver/gold multilayer to increase surface plasmon propagation length. The metal structure can be deposited on the prism using a lithographic process. Adaptations of this technique are described in co-pending international patent application PCT/GB03/03803, the content of which is hereby incorporated by reference.

The amplification reaction is carried out using conventional PCR reagents and conditions. In summary, a target polynucleotide is contacted with a polymerase enzyme, the necessary primer molecules and the various nucleic acid monomers (bases) so that incorporation of the monomers onto the target polynucleotide can occur. A polynucleotide complementary to that of the target polynucleotide is then synthesised by the polymerase. After the complementary polynucleotide is synthesised, the temperature at which the reaction is performed is increased so that the hybridisation between the complement and the target is disrupted and dissociation occurs. The target and the complement may then be used as substrates for further amplification.

The amplification reaction and the detection of the binding/interaction between the molecule and any amplified product is intended to be carried out in the same reaction vessel and in “real-time”, i.e. both amplification and detection are carried out at the same time. As the amplification reaction proceeds, the amount of amplified product can be measured. Identification of amplified products can therefore be carried out during the whole or substantially the whole of a PCR thermal cycle. As identification is of the amplified product only, spurious background measurements can be reduced or avoided.

In a preferred embodiment, the polymerase reaction is carried out within a sealed micro-flow cell. The reactants are introduced into the flow cell which is then sealed by closing input and output valves. In a preferred embodiment, an integrated pump is incorporated to maintain the reaction/PCR fluid flowing in the closed cell, thus increasing diffusion at the detection surface making detection of the amplified reaction products more favourable The reactant mixture is allowed to flow over the immobilised molecule so that amplified products can interact with the molecule, permitting the detection of the interaction and consequently the quantification of the amplification process. As the amplification reaction proceeds the increased amount of amplified product will interact with additional immobilised molecules, generating an increase in the signal detected. Detecting the signal permits the amplification reaction to be quantified.

Multiplexed reactions may be carried out by incorporating spatially separated molecules which interact specifically with one type of amplification product. For example, DNA-binding proteins may be used which contain different polynucleotide binding domains. It is therefore possible to distinguish the amplification products based on their interaction with different binding proteins. Alternatively, if polynucleotides are used as the binding molecule, they can be designed so that a range of different sequences are present at defined locations, allowing sequence specific interactions to be monitored.

Such interactions maybe measured at one particular point in the amplification cycle and compared with like points within subsequent cycles (i.e. at the same temperature in the case of thermocycling reactions). In one particular preferred embodiment, interaction data are obtained for the entire thermocycle and a “melting” curve obtained. In the case in which polynucleotides are employed as the probe molecule, such curves are of particular benefit due to their ability to more effectively distinguish between covalently hybridised duplexes and “false” positive sequences containing smaller regions of sequence homology.

The content of each of the publications referred to herein, is incorporated by reference.

The following Example illustrates the invention.

### EXAMPLE

A 50 nanometer thick and 70 micron wide gold microstructure was lithographically fabricated as outlined in co-pending international patent application PCT/GBO3/03803. The chip was then cleaned sonically in acetone and placed in an ozone cleaner for approximately 20 minutes. The chip was then assembled into the system (FIG. 1) and the excitation laser was focussed onto the leading edge of the microstructure. The laser spot was then adjusted so that optimal bulk refractive index compensation was achieved (for read-out an interference pattern is formed on a CCD camera). The assembled sensor includes a micro-flow cell, allowing samples to be injected over the surface of the sensor chip.

A thiolated oligo was obtained from QlAgen (5′-[ThiSS] TAAAACGACGGCCAGTGC-3′) after HPLC purification. A 1 μM of the solution of the thiolated oligo in 5× SET buffer was incubated overnight in the flow cell at a flow rate of 20 μl hour⁻¹. The next day the cell was washed with a flow rate of 5 μl minute⁻ with 5× SET for ten minutes, 5× SET, 0.1% SDS for 60 minutes, 5× SET for 10 minutes and 5× SET, 0.1% mercaptohexanol for 60 minutes. The flow cell was equilibrated with a flow stream of 2× SET at 5 μl minute⁻¹ and held at 20° C.

Complementary DNA (5′- GCACTGGCCGTCGTTTTA) 1 μM in 2× SET was injected into the flow stream at a rate of 5 μl minute⁻¹. Association of the complimentary sequence with the immobilised probe on the sensor surface was detected by the interoferometric system. When the fluidic cell was heated for 30 seconds (to a thermo-couple temperature of 50° C.) the double stranded DNA dissociated and the baseline returned to the previous level. 1 mM NaNO₂ was injected into the flow stream and resulted in the removal of the thiolated oligo from the sensor surface and a concominant decrease in correlation shift. This experiment therefore demonstrates the label free Surface Plasmon Resonance monitoring of polynucleotide association and dissociation with a immobilized probe molecule as a function of temperature. This is of particular use in context of real-time PCR.

We claim:

1. A method for monitoring the amplification of a plurality of different target polynucleotides in a single reaction chamber, comprising:
(i) carrying out a reaction for the amplification of a plurality of different target polynucleotides;
(ii) during the amplification reaction, contacting different amplified products with a molecule that binds to or interacts with a polynucleotide, the molecule being located in a spatially defined position or being determined via a non-linear or non-fluorescent technique; and
(iii) detecting the interaction between the amplified product and the molecule by measuring changes in applied radiation.

2. The method according to claim 1, wherein the molecule is immobilized to a support material.

3. The method according to claim 1, wherein the molecule is a polymerase enzyme.

4. The method according to claim 1, wherein the molecule is a polynucleotide, at least a portion of which is complementary to a region on an amplified product.

5. The method according to claim 4, wherein the molecule acts as a primer for the amplification reaction.

6. The method according to claim 4, wherein the molecule does not act as a primer for the amplification reaction.

7. The method according to claim 1, wherein the detecting is carried out by detection of an evanescent field.

8. The method according to claim 1, wherein the detecting is carried out by applying surface electromagnetic waves and monitoring changes in the waves.

9. The method according to claim 7, wherein the detecting is carried out by measuring changes in surface plasmon resonance.

10. The method according to claim 9, wherein the molecule comprises a metallic particle.

11. The method according to claim 1, wherein the detecting is carried out by detecting surface enhanced Raman scattering.

12. The method according to claim 4, wherein the detecting is achieved by detecting an intercalating label that binds to the hybrid formed between the amplified product and polynucleotide during the amplification reaction.

13. The method according to claim 12, wherein the intercalating label is fluorescent when bound to the hybrid.

14. The method according to claim 1, wherein the detecting is achieved by monitoring changes in electrical conductance and/or capacitance.

15. The method according to claim 1, wherein the amplification reaction occurs in a sealed micro-flow cell.

16. An apparatus for monitoring a polynucleotide amplification reaction, comprising a support material having a plurality of molecules immobilized thereon, the molecules having the ability to bind to or interact with a polynucleotide, and means for detecting changes in applied radiation.

17. The apparatus according to claim 16, further comprising a sealed micro-flow cell.

18. The apparatus according to claim 16, further comprising a pump to maintain a flow of fluid over the support material.

19. The apparatus according to claim 17, further comprising a pump to maintain a flow of fluid over the support material.


SEQUENCE LISTING (2 sequences)

SEQ ID NO: 1 - DNA, 18 bp, Artificial
  Artifical Oligonucleotide
taaaacgacggccagtgc

SEQ ID NO: 2 - DNA, 18 bp, Artificial
  Artifical Oligonucleotide
gcactggccgtcgtttta